(19) United States
(12) Patent Application Publication
(10) Pub. No.: US 2020/0102574 A1
(43) Pub. Date: Apr. 2, 2020

(54) Functional Nucleic Acid Protective Vector Based On DNA Hydrogel, Preparation Method and Application Thereof

(71) Applicant: Shanghai Jiao Tong University, Shanghai (CN)

(72) Inventors: Chuan Zhang, Shanghai (CN); Fei Ding, Shanghai (CN); Quanbing Mou, Shanghai (CN)

(21) Appl. No.: 16/621,408

(22) PCT Filed: May 23, 2018
(86) PCT No.: PCT/CN2018/088035

§ 371 Date: Dec. 11, 2019

(30) Foreign Application Priority Data
Jul. 10, 2017 (CN) 201710557313.0

(51) Int. Cl. C12N 15/85; C12N 15/64; C12N 15/113

(52) U.S. Cl. CPC C12N 15/85; C12N 15/64; C12N 15/113; C12N 2310/11; C12N 2310/3519; C12N 2330/30; C12N 2310/14

(57) ABSTRACT

The present invention belongs to the technical field of biological medicine, and specifically discloses a functional nucleic acid protective vector based on DNA hydrogels, which is self-assembled by a biodegradable high-molecular polymer with DNA-grafted side chain, a functional nucleic acid and a cross-linking agent. In aqueous solution, the DNA hydrogels of the present invention may be applied to self-assemble into particles with controllable and uniform sizes in situ at room temperature; the particles have very good stability under physiological conditions, and by wrapping functional nucleic acids in the interior DNA hydrogel, it may effectively abate the degradation by nuclease, and moreover, the prepared DNA hydrogel may effectively deliver functional nucleic acids to cytoplasm without any kation, virus or other transfection reagents, thus achieving therapeutic effects, as well as avoiding toxic and side effects caused by the introduced kation, virus or other transfection reagents.

### TECHNICAL FIELD

The present invention belongs to the technical field of biological medicine, and particularly relates to a functional nucleic acid protective vector based on DNA hydrogels, preparation method and application thereof.

### BACKGROUND

Gene therapy is an important way to treat many intractable diseases. However, there are still many challenges, e.g., poor stability, easy degradation, difficulty in cellular uptake, low bioavailability, unreasonable body distribution, short half-life period of body circulation, etc. in the nucleic acid drugs associated with gene therapy (Adv. Drug Delivery Rev., 2009, 8, 129-138.), and these problems limit the clinical application of nucleic acid drugs tremendously. In recent years, with the development of nanotechnology, people have developed various kinds of vectors for the delivery of nucleic acid drugs. The vector for delivering nucleic acid drugs is mainly divided into two types, one is a viral vector, and another one is a non-viral vector. The viral vector can efficiently transfect the cells with functional nucleic acids, but their immunogenicity and potential genotoxicity severely constrain their application (Gene Ther., 2008, 15, 1500-1510.). Generally, the non-viral vector is constituted by positively-charged cationic polymers, such as, PEI, micelle (J. Am. Chem. Soc., 2015, 137, 15217-15224.), lipidosome (J. Am. Chem. Soc., 2015, 137, 6000-6010.), these cationic polymers bond with negatively-charged functional nucleic acids via electrostatic interaction, thus delivering functional nucleic acids. The gene silencing efficacy of this strategy is associated with the properties of the cationic polymers; when cationic polymers carry more positive charges, the electrostatic interaction with functional nucleic acids is stronger, thereby the gene silencing efficacy is better, otherwise, the efficacy is worse (J. Controlled Release, 2007, 123, 1-10.). However, when cationic polymers carry more positive charges, it causes severe toxic and side effects (Adv. Drug Delivery Rev., 2012, 64, 1717-1729.). Therefore, it is urgent to develop a new delivery strategy that not only efficiently silences pathogenic genes, but also greatly reduces side effects. With the development of DNA nanotechnology, a kind of material without the transfection of cationic polymers has aroused extensive concerns, including spherical nucleic acids (SNA) and DNA origmi. SNA is a kind of spherical nucleic acid formed by regarding nanoparticles as its nucleus and modifying high-density single/double-stranded nucleic acids on its surface; different from common single-stranded nucleic acid, SNA can identify vectors on the surface of cells, thus triggering endocytosis (J. Am. Chem. Soc., 2009, 2072-2073.), which achieves the delivery of functional nucleic acids without the use of cationic transfection reagents. Similarly, as a kind of nano particle having a 3D nanostructure, the DNA origami can also interact with receptors on the surface of cells to trigger endocytosis (Nat. nanotech., 2012, 7, 389-393; Angew. Chem. Int. Ed., 2014, 53, 7745-7750.). But the functional nucleic acids loaded by SNA or DNA origami are always exposed on the surface of nanoparticles. As such, the loaded functional nucleic acids may be not protected efficiently, impeding its use in clinic practices.

### SUMMARY

The first objective of the present invention is to provide a functional nucleic acid protective vector based on DNA hydrogels, so as to achieve the efficient delivery of functional nucleic acids, as well as to solve the technical problems, such as, human immune responses, genotoxicity, inflammation and toxicity of human body and some other symptoms caused by the existing virus capsid carriers or cationic polymers taken in the existing delivery technology.

The second objective of the present invention is to provide a preparation method of the above functional nucleic acid protective vector based on DNA hydrogels with controllable size.

The third objective of the present invention is to provide an application of the above functional nucleic acid protective vector based on DNA hydrogels in the preparation of nucleic acid drugs for disease treatment based on gene therapy.

The technical solution of the present invention is detailed as follows:

A functional nucleic acid protective vector based on DNA hydrogels is self-assembled by a biodegradable polymer with DNA-grafted side chain, a functional nucleic acid and a cross-linking agent.

Preferably, the biodegradable high-molecular polymer with DNA-grafted side chain is obtained by conjugating a biodegradable polymer with azide groups on its side chain with diphenylcyclooctyne-modified DNA (DBCO-DNA).

Preferably, the biodegradable polymer is polymerized by one of the following monomers, namely, polycaprolactone, polyphosphoester, polylactic acid, polylactic acid-glycolic acid copolymer and polypeptide, but not limited to the above biodegradable polymers.

Preferably, NDA bases on the biodegradable high-molecular polymer with DNA-grafted side chain are selected randomly, and the number of the bases is more than 8; the cross-linking agent consists of two partial-complementary DNA chains, where, the base sequence of the complementary portion is random, and the number of the basic groups is more than 12; DNA in the non-complementary portion may be paired with DNA of the side chain of the degradable high-molecular polymer.

Preferably, the functional nucleic acid is one of siRNA, mRNA, plasmid, non-coding RNA, anti sense oligonucleotide or Cas9-sgRNA.

Preferably, when siRNA is chosen as the functional nucleic acid, it also serves as a cross-linking agent, which contains a segment of nucleotide sequence capable of pairing with DNA brushes (side chains) grafted on the degradable polymer additionally on the tail of the siRNA antisense strand and sense strand respectively.

Preferably, when other functional nucleic acid is chosen, there is a segment of nucleotide sequence capable of pairing with DNA brushes (side chains) grafted on the degradable polymer additionally on one tail of the functional nucleic acid.

The present invention further discloses a preparation method of the above functional nucleic acid protective vector based on DNA hydrogels, including the following steps:

(1) a biodegradable polymer with azide groups on its side chain is synthesized by ring opening polymerization, then the degradable polymer reacts with DBCO-DNA via copper-free click chemistry to obtain a biodegradable high-molecular polymer with DNA-grafted side chain;

(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other. After that, the cross-linking agent is added to pair with the biodegradable high-molecular polymer with DNA-grafted side chain completely to obtain DNA hydrogel solution.

Preferably, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

More preferably, when the molar ratio of the DNA in the degradable high-molecular polymer to the cross-linking agent siRNA is 8:1, 7:1, 6:1, 5:1, 4:1, 3:1, 2:1, the size of the prepared DNA hydrogel is respectively 75 nm, 100 nm, 120 nm, 200 nm, 360 nm, 650 nm, 1.2 μm.

When the molar ratio of the DNA in the degradable high-molecular polymer to the cross-linking agent (excepting for siRNA) is 8:1, 7:1, 6:1, 5:1, 4:1, 3:1, 2:1, the size of the prepared DNA hydrogel is respectively 80 nm, 120 nm, 140 nm, 210 nm, 380 nm, 660 nm, 1.3 μm.

The present invention further discloses an application of the above functional nucleic acid protective vector based on DNA hydrogels in the preparation of nucleic acid drugs for disease treatment based on gene therapy

Compared with the prior art, beneficial effects of the present invention are detailed as follows:

I. The functional nucleic acid protective vector based on DNA hydrogels of the present invention may be in situ self-assembled in aqueous solution, and may protect functional nucleic acids during its delivery, abating the degradation thereof by nuclease, thus finishing the delivery of the functional nucleic acids, moreover, as delivery vectors, the degradable high-molecular polymer and NDA do not cause toxic and side effects;

II. The preparation method of the functional nucleic acid protective vector based on DNA hydrogels of the present invention may be applied to self-assemble into particles with controllable and uniform sizes in situ at room temperature; the particles have very good stability under physiological conditions, and by wrapping functional nucleic acids in the interior DNA hydrogel, it may effectively abate the degradation by nuclease, moreover, and the prepared DNA hydrogel may effectively deliver functional nucleic acids to cytoplasm without any kation, virus or other transfection reagents, thus achieving therapeutic effects, as well as avoiding toxic and side effects caused by the introduced kation, virus or other transfection reagents.

Certainly, the implementation of any one of the products in the present invention need not necessarily achieve all the above advantages at the same time.

### BRIEF DESCRIPTION OF THE DRAWINGS

FIG. 1 is a schematic diagram showing a preparation route of a DNA hydrogel A in embodiment 1;

FIG. 2 is a ¹H NMR spectrogram of a polymer 1 in embodiment 1;

FIG. 3 is a ¹H NMR spectrogram of a polymer 2 in embodiment 1;

FIG. 4 is a data graph showing gel permeation chromatography of the polymers 1 and 2 in embodiment 1;

FIG. 5 is a denaturing gel electrophoretogram of a polymer 3 in embodiment 1;

FIG. 6 is a 0.5% agaroseelectrophoretogram of the DNA hydrogel A in embodiment 1;

FIG. 7 is a data graph showing hydrodynamic diameter of the DNA hydrogel A prepared in step 4 and the polymer in embodiment 1;

FIG. 8 is an atomic force microscope (AFM) photograph of the polymer 3 in embodiment 1;

FIG. 9 is an AFM photograph of a DNA hydrogel A8-1 in embodiment 1;

FIG. 10 is an AFM photograph of a DNA hydrogel A7-1 in embodiment 1;

FIG. 11 is an AFM photograph of a DNA hydrogel A6-1 in embodiment 1;

FIG. 12 is an AFM photograph of a DNA hydrogel A5-1 in embodiment 1;

FIG. 13 is an AFM photograph of a DNA hydrogel A4-1 in embodiment 1;

FIG. 14 is an AFM photograph of a DNA hydrogel A3-1 in embodiment 1;

FIG. 15 is an AFM photograph of a DNA hydrogel A2-1 in embodiment 1;

FIG. 16 is a schematic diagram showing a preparation route of a DNA hydrogel B loading antisense in embodiment 2;

FIG. 17 is a schematic diagram showing a preparation route of a DNA hydrogel C loading with siRNA in embodiment 3;

FIG. 18 is a 0.5% agaroseelectrophoretogram of the DNA hydrogel A6-1 in embodiment 1 incubated with DMEM medium containing 10% FBS at different time;

FIG. 19 is a 10% denaturing gel electrophoretogram of the DNA hydrogel A6-1 incubated with different concentrations of RNA enzyme;

FIG. 20 is a schematic diagram showing the apoptosis of tumor cells induced by the siRNA-loaded DNA hydrogel A6-1 (the siRNA can target and silence PLK1 protein) in embodiment 1.

### DETAILED DESCRIPTION

Hereafter, the present invention will be further described with reference to the detailed embodiments below. It should be understood that these embodiments are used for construing the invention only, but not limiting its protection scope. Improvements and adjustments made by those skilled in the art according to the present invention in practical use are still within the protection scope of the present invention.

### Embodiment 1

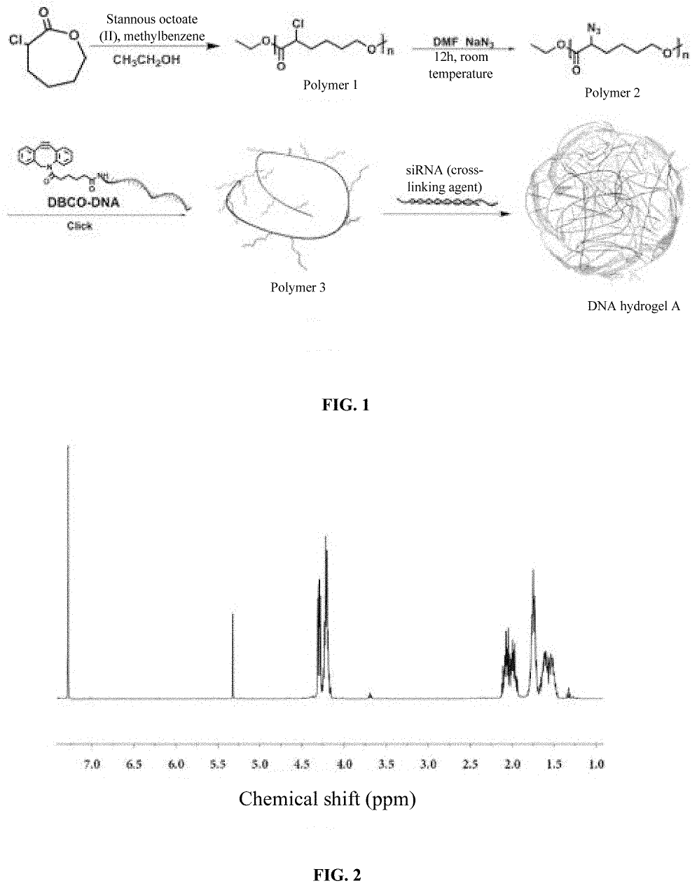

The preparation route of the DNA hydrogel A in embodiment 1 was shown in FIG. 1, and its specific steps were as follows:

1.1 Synthesis of a Polymer 1

2-chloro-caprolactone (700.0 mg) was dissolved into 15 ml anhydrous methylbenzene, and to water in the solution was removed by azeotropy of methylbenzene and water. Thereafter, the solution was firstly heated to 70° C. under N₂. Then one drop of tin (II) octanoate and dry ethanol (17 mg) was added. The temperature was maintained at 70° C. for another 20 min, then raised to 130° C. for 4 hours. The crude polymer was dissolved in dichloromethane and precipitated into ice ether. The product was purified by repeated dissolution in dichloromethane and ice ether precipitation thrice, the resulting white powder dried under reduced pressure to afford 500 mg polymer 1, and the productive rate was 69.7%.

The ¹H NMR spectrogram of the polymer 1 is shown in FIG. 2, test solvent is CDCl₃ and each of proton peaks is attributed below: δ(ppm): 4.32-4.27 (m, 64H, ClCH), 4.26-4.15 (m, 128H, OCH₂), 2.14-1.92 (m, 135H, CH₂), 1.82-1.69 (m, 134H, CH₂), 1.68-1.44 (m, 140H, CH₂), 1.35-1.30 (t, 3H, CH₃). The number-average molecular weight Mn of polymer 1 is 11300, mass-average molecular weight Mw is 15304, as shown in FIG. 4.

1.2 Synthesis of a Polymer 2

A mixture of polymer 1 (500.0 mg) and NaN₃ (400 mg) in 10 mL anhydrous N,N′-dimethylformamide was stirred at 25° C. for 12 h. After removed the N,N′-dimethylformamide by reduced pressure distillation, 5 mL methylbenzene was added, and the remaining NaN₃ was removed by centrifugation (4000 rmp) for 20 min. The polymer was recovered by precipitation in ice ether. After thoroughly washing with ice ether three times, the white powder was collected by vacuum filtration and dried under reduced pressure to afford 350 mg polymer 2, and the productive rate was 68.4%.

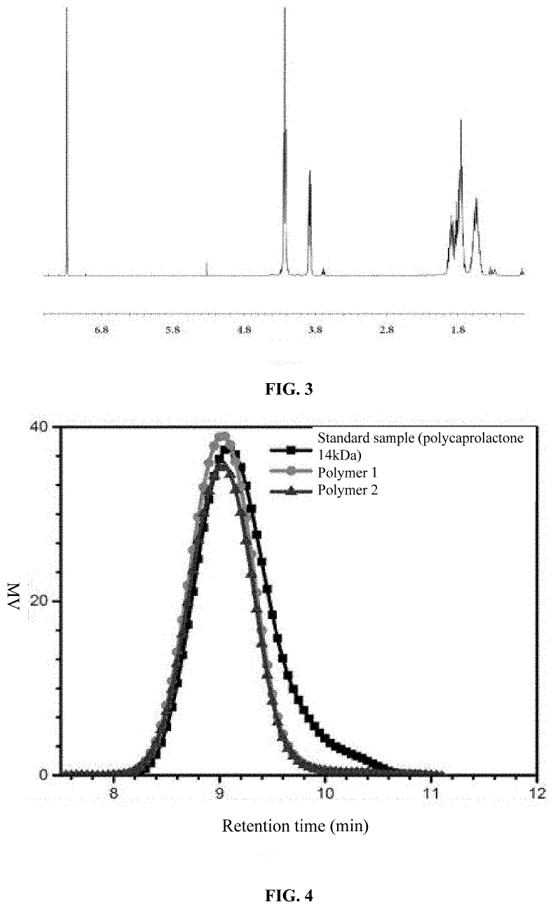

The ¹H NMR spectrogram of the polymer 2 is shown in FIG. 3, test solvent is CDCl₃ and each of proton peaks is attributed below: δ(ppm): 4.29-4.17 (m, 131H, OCH₂), 3.91-3.84 (m, 64H, N₃CH), 1.96-1.67 (m, 281H, CH₂CH₂), 1.64-1.44 (m, 139H, CH₂), 1.36-1.32 (t, 3H, CH₃). The number-average molecular weight Mn of polymer 2 is 11217, mass-average molecular weight Mw is 15305, as shown in FIG. 4.

1.3 Synthesis of a Polymer 3

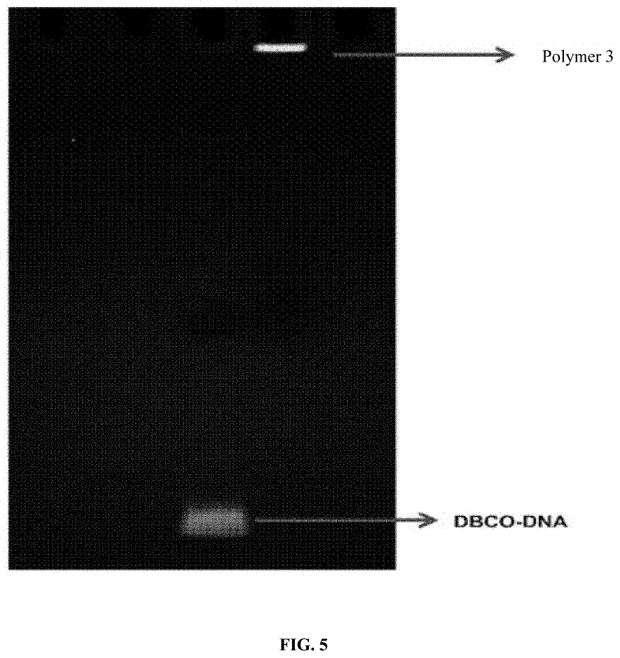

The polymer 2 (0.132 mg) and DBCO-DNA (3.3 mg) were dissolved into 500 μl dimethyl sulfoxide, and the resulting solution was stirred for 24 h at 50° C., Thereafter, the dimethyl sulfoxide was removed by dialysis, the unreacted DBCO-DNA was removed by centrifugation via a 50000 Da ultra-filtration centrifugal tube. The obtained solution was quantified by measuring the ultraviolet-visible absorption at 260 nm to calculate the gross DNA of the polymer 3. It can be seen from 10% denaturing PAGE gel electrophoresis that the polymer 3 contains a large amount of DNA, resulting in very slow electrophoretic velocity, as shown in FIG. 5.

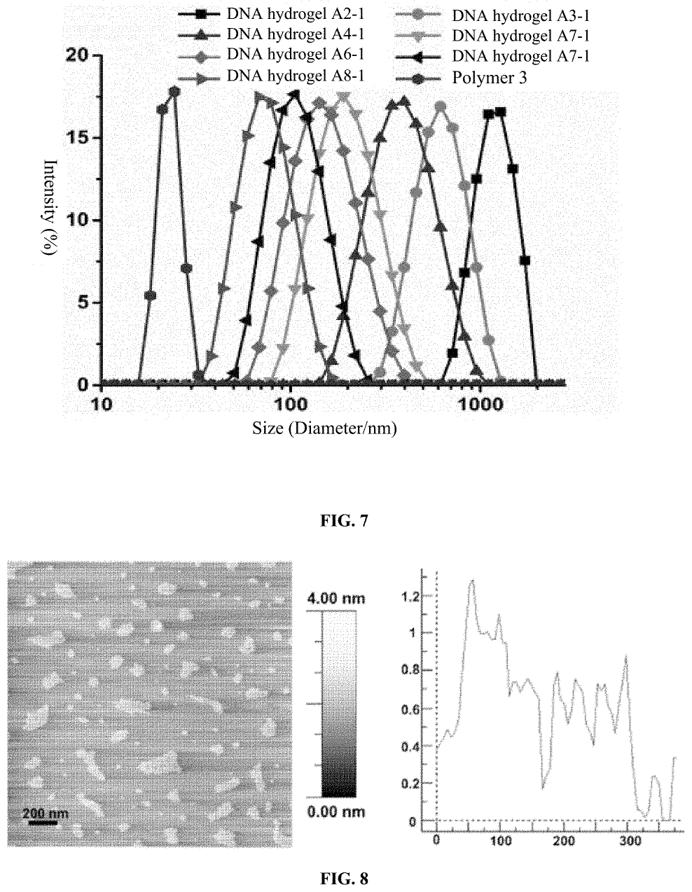

The hydrodynamic diameter of the degradable high-molecular polymer 3 with Cl-grafted side chain prepared by the step 3 is shown in FIG. 7, and the average particle hydrodynamic diameter of the polymer 3 is 21 nm. The AFM photograph is shown in FIG. 8, the average particle size of the polymer 3 is 100 nm, and the average height is 0.7 nm.

1.4 Synthesis of a DNA Hydrogel A

The influence of the different molar ratio of DNA grafted on the side chain of the polymer 3 to the siRNA on the particle size of the DNA hydrogel was specifically studied in this embodiment, so as to indicate that the particle size of the DNA hydrogel may be controlled by adjusting the molar ratio of the above two, thus a specific particle size of the DNA hydrogel may be selected according to experiment demands in the future animal experiment evaluation.

1.4.1 When the molar ratio of the DNA grafted on the side chain of the polymer 3 to the added siRNA was 8:1, the both two were mixed well and placed for 1 h at room temperature to obtain a DNA hydrogel A8-1.

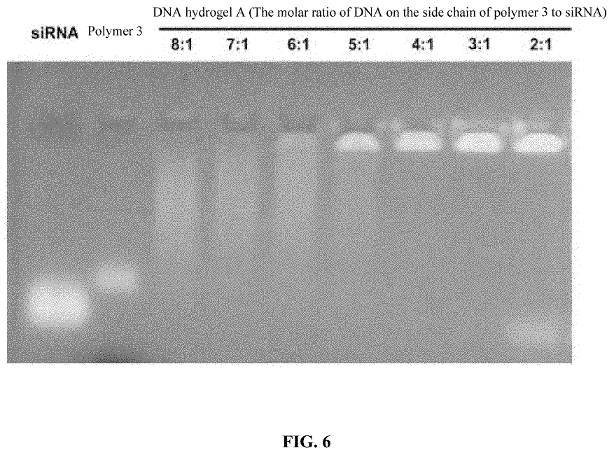

The prepared DNA hydrogel A8-1 showed dispersed bands in 0.5% agarose gel electrophoresis, moreover, the bands may completely enter into the 0.5% agarose gel, indicating that the DNA hydrogel A8-1 is less than 200 nm, as shown in FIG. 6.

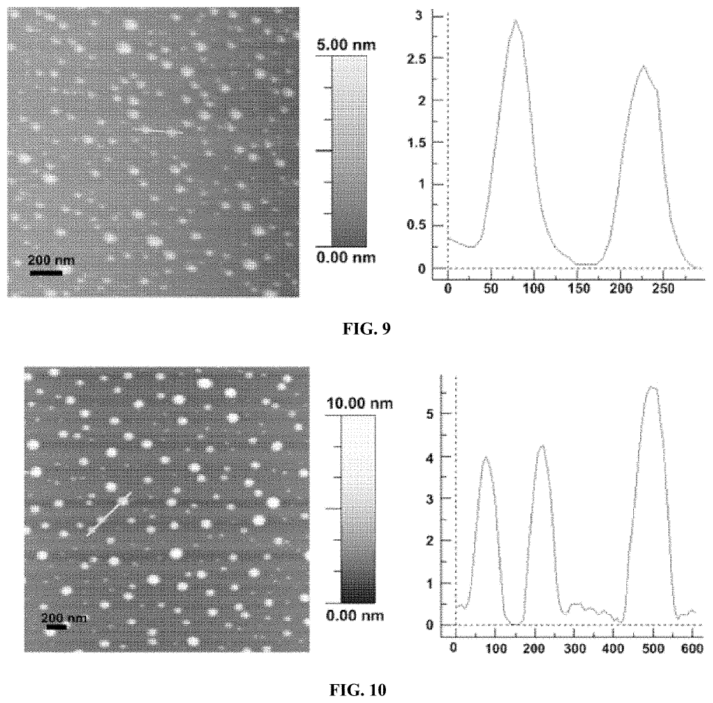

The hydrodynamic diameter of the prepared DNA hydrogel A8-1 is show in FIG. 7, and the average hydrodynamic diameter of the DNA hydrogel A8-1 is 75 nm. The AFM photograph is shown in FIG. 9, the average particle size of the DNA hydrogel A8-1 is 85 nm, and the average height is 2.5 nm.

1.4.2 When the molar ratio of the DNA grafted on the side chain of the polymer 3 to the added siRNA was 7:1, the both two were mixed well and placed for 1 h at room temperature to obtain a DNA hydrogel A7-1.

The prepared DNA hydrogel A7-1 showed dispersed bands in 0.5% agarose gel electrophoresis, moreover, the bands may completely enter into the 0.5% agarose gel, indicating that the DNA hydrogel A7-1 is less than 200 nm, as shown in FIG. 6.

The hydrodynamic diameter of the prepared DNA hydrogel A7-1 is show in FIG. 7, and the average hydrodynamic diameter of the DNA hydrogel A7-1 is 100 nm. The AFM photograph is shown in FIG. 10, the average particle size of the DNA hydrogel A7-1 is 110 nm, and the average height is 4.5 nm.

1.4.3 When the molar ratio of the DNA grafted on the side chain of the polymer 3 to the added siRNA was 6:1, the both two were mixed well and placed for 1 h at room temperature to obtain a DNA hydrogel A6-1.

The prepared DNA hydrogel A6-1 showed dispersed bands in 0.5% agarose gel electrophoresis, moreover, the strips may completely enter into the 0.5% agarose gel, indicating that the DNA hydrogel A6-1 is less than 200 nm, as shown in FIG. 6.

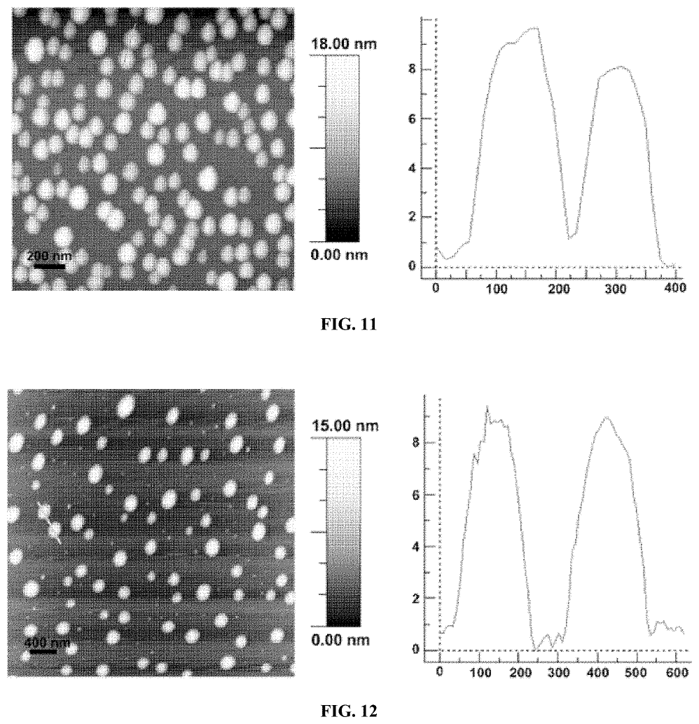

The hydrodynamic diameter of the prepared DNA hydrogel A6-1 is show in FIG. 7, and the average hydrodynamic diameter of the DNA hydrogel A6-1 is 120 nm. The AFM photograph is shown in FIG. 11, the average particle size of the DNA hydrogel A6-1 is 140 nm, and the average height is 8 nm.

1.4.4 When the molar ratio of the DNA grafted on the side chain of the polymer 3 to the added siRNA was 5:1, the both two were mixed well and placed for 1 h at room temperature to obtain a DNA hydrogel A5-1.

The prepared DNA hydrogel A5-1 showed dispersed bands in 0.5% agarose gel electrophoresis, moreover, there are bands in loading well and agarose gel, indicating that the size of the DNA hydrogel A5-1 may be more than 200 nm or less than 200 nm, as shown in FIG. 6.

The hydrodynamic diameter of the prepared DNA hydrogel A5-1 is show in FIG. 6, and the average hydrodynamic diameter of the DNA hydrogel A6-1 is 200 nm. The AFM photograph is shown in FIG. 12, the average particle size of the DNA hydrogel A5-1 is 240 nm, and the average height is 9 nm.

1.4.5 When the molar ratio of the DNA grafted on the side chain of the polymer 3 to the added siRNA was 4:1, the both two were mixed well and placed for 1 h at room temperature to obtain a DNA hydrogel A4-1.

The prepared DNA hydrogel A4-1 showed single band in 0.5% agarose gel electrophoresis, moreover, the band may be completely stuck in the loading well of the agarose gel, indicating that the size of DNA hydrogel A4-1 is more than 200 nm, as shown in FIG. 6.

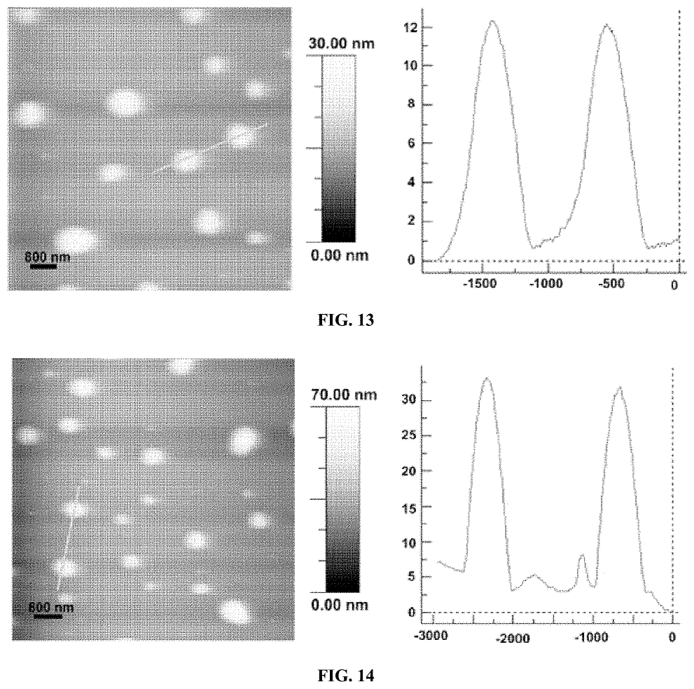

The hydrodynamic diameter of the prepared DNA hydrogel A4-1 is show in FIG. 7, and the average hydrodynamic diameter of the DNA hydrogel A4-1 is 360 nm. The AFM photograph is shown in FIG. 13, the average particle size of the DNA hydrogel A4-1 is 400 nm, and the average height is 12 nm.

1.4.6 When the molar ratio of the DNA grafted on the side chain of the polymer 3 to the added siRNA was 3:1, the both two were mixed well and placed for 1 h at room temperature to obtain a DNA hydrogel A3-1.

The prepared DNA hydrogel A3-1 showed single band in 0.5% agarose gel electrophoresis, moreover, the band may be completely stuck in the loading well of the agarose gel, indicating that the size of DNA hydrogel A3-1 is more than 200 nm, as shown in FIG. 6.

The hydrodynamic diameter of the prepared DNA hydrogel A3-1 is show in FIG. 7, and the average hydrodynamic diameter of the DNA hydrogel A3-1 is 650 nm. The AFM photograph is shown in FIG. 14, the average particle size of the DNA hydrogel A3-1 is 700 nm, and the average height is 26 nm.

1.4.7 When the molar ratio of the DNA grafted on the side chain of the polymer 3 to the added siRNA was 2:1, the both two were mixed well and placed for 1 h at room temperature to obtain a DNA hydrogel A2-1.

The prepared DNA hydrogel A2-1 showed single band in 0.5% agarose gel electrophoresis, moreover, the band may be completely stuck in the loading well of the agarose gel, indicating that the size of DNA hydrogel A2-1 is more than 200 nm, as shown in FIG. 6.

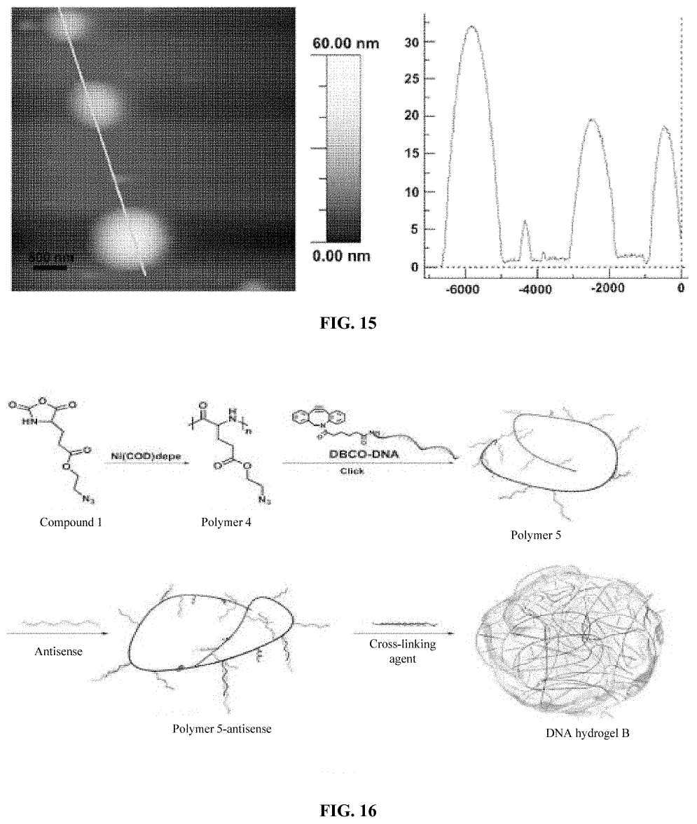

The hydrodynamic diameter of the prepared DNA hydrogel A2-1 is show in FIG. 7, and the average hydrodynamic diameter of the DNA hydrogel A2-1 is 1.1 The AFM photograph is shown in FIG. 15, the average particle size of the DNA hydrogel A2-1 is 1.3 and the average height is 25 nm.

### Embodiment 2

The preparation route of the DNA hydrogel B in embodiment 2 was shown in FIG. 16, and its specific steps were as follows:

2.1 Synthesis of a Polymer 4

A compound 1 (700.0 mg) was dissolved into 20 ml anhydrous N, N′-dimethylformamide in a glove box, and 400 μl newly-prepared Ni(COD)depe was rapidly added. The solution color turned from pale yellow to luminous yellow quickly. The mixtures were stirred for 12 h at room temperature. Thereafter, the N, N′-dimethylformamide was removed by reduced pressure distillation, and the crude polymer was dissolved in dichloromethane and precipitated into ice ether. The product was purified by repeated dissolution in dichloromethane and ice ether precipitation thrice. The resulting white power dried under reduced pressure at room temperature to afford 600 mg polymer 4, and the productive rate was 85.7%.

2.2 Preparation of a Polymer 5

The polymer 4 (0.13 mg) and DBCO-DNA (2.3 mg) were dissolved into 500 μl dimethyl sulfoxide, and the resulting solution was stirred for 24 h at 50° C. Thereafter, dimethyl sulfoxide was removed by dialysis, and the unreacted DBCO-DNA was removed by centrifugation via a 50000 Da ultra-filtration centrifugal tube. The obtained solution was quantified by measuring at 260 nm to calculate the gross DNA of the polymer 4.

The average particle hydrodynamic diameter of the polymer 5 prepared by the step was 25 nm.

2.3 Preparation of a Polymer 5-Antisense DNA Conjugate

The polymer 5 and antisense DNA were mixed into aqueous solution (when the molar ratio of the DNA grafted on the side chain of polymer 4 to the added antisense was more than 2) for pairing fully, thus forming the polymer 5-antisense DNA conjugate.

2.4 Synthesis of a DNA Hydrogel B

The influence of the different molar ratio of DNA conjugated on the side chain of polymer 5 to the added cross-linking agent DNA linker on the particle size of the DNA hydrogel was specifically studied in this embodiment, so as to indicate that the particle size of the DNA hydrogel may be controlled by adjusting the molar ratio of the above two.

2.4.1 When the molar ratio of the DNA grafted on the side chain of the polymer 5 to the added cross-linking agent DNAlinker was 8:1, the both two were mixed well and placed for 1 h at room temperature to obtain a DNA hydrogel B8-1.

The average particle hydrodynamic diameter of the prepared DNA hydrogel B8-1 was 70 nm.

2.4.2 When the molar ratio of the DNA grafted on the side chain of the polymer 5 to the added cross-linking agent DNAlinker was 7:1, the both two were mixed well and placed for 1 h at room temperature to obtain a DNA hydrogel B7-1.

The average particle hydrodynamic diameter of the prepared DNA hydrogel B7-1 was 100 nm.

2.4.3 When the molar ratio of the DNA grafted on the side chain of the polymer 5 to the added cross-linking agent DNAlinker was 6:1, the both two were mixed well and placed for 1 h at room temperature to obtain a DNA hydrogel B6-1.

The average particle hydrodynamic diameter of the prepared DNA hydrogel B6-1 was 135 nm.

2.4.4 When the molar ratio of the DNA grafted on the side chain of the polymer 5 to the added cross-linking agent DNAlinker was 5:1, the both two were mixed well and placed for 1 h at room temperature to obtain a DNA hydrogel B5-1.

The average particle hydrodynamic diameter of the prepared DNA hydrogel B5-1 was 200 nm.

2.4.5 When the molar ratio of the DNA grafted on the side chain of the polymer 5 to the added cross-linking agent DNAlinker was 4:1, the both two were mixed well and placed for 1 h at room temperature to obtain a DNA hydrogel B4-1.

The average particle hydrodynamic diameter of the prepared DNA hydrogel B4-1 was 340 nm.

2.4.6 When the molar ratio of the DNA grafted on the side chain of the polymer 5 to the added cross-linking agent DNAlinker was 3:1, the both two were mixed well and placed for 1 h at room temperature to obtain a DNA hydrogel B3-1.

The average particle hydrodynamic diameter of the prepared DNA hydrogel B3-1 was 600 nm.

2.4.7 When the molar ratio of the DNA grafted on the side chain of the polymer 5 to the added cross-linking agent DNAlinker was 2:1, the both two were mixed well and placed for 1 h at room temperature to obtain a DNA hydrogel B2-1.

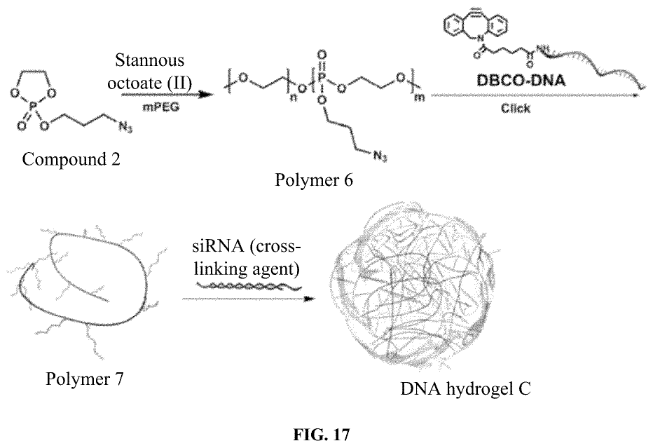

The average particle hydrodynamic diameter of the prepared DNA hydrogel B2-1 was 1.2 Embodiment 3 The preparation route of the DNA hydrogel C in embodiment 3 was shown in FIG. 17, and its specific steps were as follows:

3.1 Preparation of a Polymer 6

Polyethylene glycol monomethyl ether (100.0 mg) was dissolved into 15 ml anhydrous methylbenzene, and trace water in the solution was removed by azeotropy of methylbenzene and water, and the residual methylbenzene was removed by reduced pressure. Thereafter, the solution was transferred to the glove box, and the compound 2 (357 mg) and a drop of stannous octoate (II) was dissolved into 10 ml dried tetrahydrofuran. Then, the mixtures were stirred for 3 h at 35° C. The crude polymer was dissolved in methyl alcohol and precipitated into ice ether. The product was purified by repeated dissolution in methyl alcohol and ice ether precipitation thrice. The resulting white powder dried under reduced pressure to afford 232 mg polymer 6, and the productive rate was 50.8%.

3.2 Preparation of a Polymer 7

The polymer 6 (0.110 mg) and DBCO-DNA (2.8 mg) were dissolved into 500 μl dimethyl sulfoxide, and the resulting solution was stirred for 24 h at 50° C. Thereafter, dimethyl sulfoxide was removed by dialysis, the unreacted DBCO-DNA was removed by centrifugation via a 50000 Da ultra-filtration centrifugal tube. The obtained solution was quantified by measuring at 260 nm to calculate the gross DNA of the polymer 7.

The average particle hydrodynamic diameter of the polymer 7 prepared by the step was 18 nm.

3.3 Synthesis of a DNA Hydrogel C

The influence of the different molar ratio of DNA conjugated on the side chain of polymer 7 to the added siRNA on the particle size of the DNA hydrogel was specifically studied in this embodiment, so as to indicate that the particle size of the DNA hydrogel may be controlled by adjusting the molar ratio of the above two.

3.3.1 When the molar ratio of the DNA grafted on the side chain of the polymer 7 to the added siRNA was 8:1, the both two were mixed well and placed for 1 h at room temperature to obtain a DNA hydrogel C8-1.

The average particle hydrodynamic diameter of the prepared DNA hydrogel C8-1 was 67 nm. 3.3.2 When the molar ratio of the DNA grafted on the side chain of the polymer 7 to the added siRNA was 7:1, the both two were mixed well and placed for 1 h at room temperature to obtain a DNA hydrogel C7-1.

The average particle hydrodynamic diameter of the prepared DNA hydrogel C7-1 was 95 nm.

3.3.3 When the molar ratio of the DNA grafted on the side chain of the polymer 7 to the added siRNA was 6:1, the both two were mixed well and placed for 1 h at room temperature to obtain a DNA hydrogel C6-1.

The average particle hydrodynamic diameter of the prepared DNA hydrogel B6-1 was 120 nm.

3.3.4 When the molar ratio of the DNA grafted on the side chain of the polymer 7 to the added siRNA was 5:1, the both two were mixed well and placed for 1 h at room temperature to obtain a DNA hydrogel C5-1.

The average particle hydrodynamic diameter of the prepared DNA hydrogel C5-1 was 190 nm.

3.4.5 When the molar ratio of the DNA grafted on the side chain of the polymer 7 to the added siRNA was 4:1, the both two were mixed well and placed for 1 h at room temperature to obtain a DNA hydrogel C4-1.

The average particle hydrodynamic diameter of the prepared DNA hydrogel C4-1 was 340 nm.

3.4.6 When the molar ratio of the DNA grafted on the side chain of the polymer 7 to the added siRNA was 3:1, the both two were mixed well and placed for 1 h at room temperature to obtain a DNA hydrogel C3-1.

The average particle hydrodynamic diameter of the prepared DNA hydrogel C3-1 was 600 nm.

3.4.7 When the molar ratio of the DNA grafted on the side chain of the polymer 7 to the added siRNA was 2:1, the both two were mixed well and placed for 1 h at room temperature to obtain a DNA hydrogel C2-1.

The average particle hydrodynamic diameter of the prepared DNA hydrogel C2-1 was 1.0 μm.

### Embodiment 4

The DNA hydrogel of the present invention may stably exist in a DMEM medium containing 10% FBS.

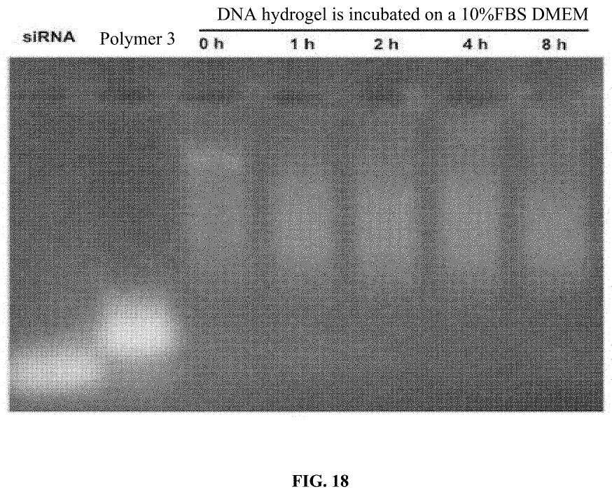

The DNA hydrogel A6-1 prepared in step 4 of embodiment 1 was incubated with DMEM containing 10% FBS for 1 h, 2 h, 4 h and 8 h at 37° C. respectively, and 0.5% agarose gel electrophoresis was used for analysis, and its results were shown in FIG. 18. When it was incubated to 8 h, there was no band of polymer 3 and siRNA in the bands escaping from the agarose gel, moreover, there was almost no band shift for the incubated DNA hydrogel A6-1 compared to that of the untreated one, indicating that the DNA hydrogel A6-1 may stably exist in DMEM medium containing 10% FBS.

### Embodiment 5

The DNA hydrogel of the present invention may effectively slow down siRNA degradation by a RNA enzyme.

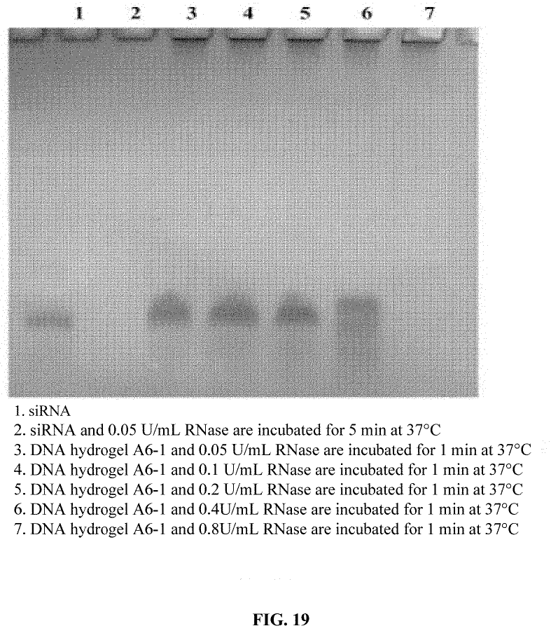

The DNA hydrogel A6-1 prepared in step 4 of embodiment 1 was incubated with different concentration of RNA enzymes (0.05 U/mL, 0.1 U/mL, 0.2 U/mL, 0.4 U/mL, 0.8 U/mL) for 1 h at 37° C., and the treated samples were analyzed by 10% denaturing gel electrophoresis. The results were shown in FIG. 19. In a control group, the naked siRNA was completely degraded by 0.05 U/mL RNA enzyme for 5 min at 37° C. In comparison, the siRNA embedded in the DNA hydrogel A6-1 only partially degraded even incubated with 0.4 U/mL RNA enzyme for 1 h, indicating that DNA hydrogel may effectively slow down RNA enzyme-mediated siRNA degradation.

### Embodiment 6

The DNA hydrogel of the present invention may inhibit the proliferation of tumors by gene silencing, subsequently inducing tumor apoptosis.

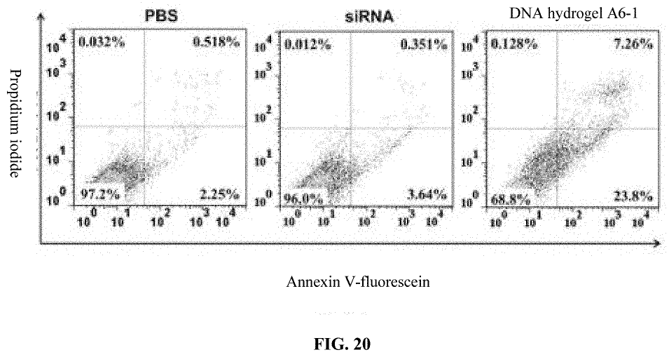

A Polo-like kinase 1 (PLK1) was selected as the oncogenic target for gene silencing. The DNA hydrogel A6-1 prepared in step 4 of embodiment 1 and MDA-MB-231 cells were co-cultured for 72 h, then apoptosis test was conducted by an AnnexinV-FITC/PI method. The results were shown in FIG. 20; the DNA hydrogel A6-1 of the siRNA loading a silencing PLK1 protein showed a very good capacity to induce cancer cell apoptosis, indicating that the DNA hydrogel has potential application value in the treatment of malignant tumors.

The preferred embodiments of the present invention disclosed above are only used to help describing the present invention. The preferred embodiments do not describe all the details specifically, nor limit that the invention is the specific implementation mode only. Apparently, lots of modifications and changes may be made according to the content of the description. The embodiments were chosen and described in the description, so as to explain the principles of the present invention and its practical applications and to thereby enable those skilled in the art to understand and utilize the present invention better. The present invention is only limited by the claim and its full scope and equivalents thereof.

1. A functional nucleic acid protective vector based on DNA hydrogels, wherein the vector is self-assembled by a biodegradable high-molecular polymer with DNA-grafted side chain, a functional nucleic acid and a cross-linking agent.

2. The functional nucleic acid protective vector based on DNA hydrogels according to claim 1, wherein the biodegradable high-molecular polymer with DNA-grafted side chain is obtained by conjugating a biodegradable polymer with azide groups on its side chain with diphenylcyclooctyne-modified DNA.

3. The functional nucleic acid protective vector based on DNA hydrogel according to claim 2, wherein the degradable polymer is polymerized by one of the following monomers, namely, polycaprolactone, polyphosphoester, polylactic acid, polylactic acid-glycolic acid copolymer and polypeptide.

4. The functional nucleic acid protective vector based on DNA hydrogels according to claim 1, wherein NDA bases on the biodegradable high-molecular polymer with DNA-grafted side chain are selected randomly, but the number of the bases is more than 8; the cross-linking agent consists of two partial-complementary DNA chains, wherein, the base sequence of the complementary portion is random, but the number of the bases is more than 12; DNA in the non-complementary portion may be paired with DNA grafted on the side chain of the degradable high-molecular polymer.

5. The functional nucleic acid protective vector based on DNA hydrogels according to claim 4, wherein the functional nucleic acid is one of siRNA, mRNA, plasmid, non-coding RNA, antisense oligonucleotide or Cas9-sgRNA.

6. The functional nucleic acid protective vector based on DNA hydrogels according to claim 5, wherein, when siRNA is chosen as the functional nucleic acid, it also serves as a cross-linking agent, which contains a segment of nucleotide sequence capable of pairing with DNA side chains grafted on the biodegradable high-molecular polymer additionally on the tail of the siRNA antisense strand and sense strand respectively.

7. The functional nucleic acid protective vector based on DNA hydrogels according to claim 5, wherein, when other functional nucleic acid is chosen, there is a segment of nucleotide sequence capable of pairing with DNA side chains grafted on the biodegradable high-molecular polymer additionally on one tail of the functional nucleic acid.

8. A preparation method of the functional nucleic acid protective vector based on DNA hydrogels according to claim 1, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

9. The preparation method of the functional nucleic acid protective vector based on DNA hydrogel according to claim 8, wherein, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

10. An application of the functional nucleic acid protective vector based on DNA hydrogel according to claim 1 in the preparation of nucleic acid drugs for disease treatment based on gene therapy.

11. A preparation method of the functional nucleic acid protective vector based on DNA hydrogels according to claim 2, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

12. The preparation method of the functional nucleic acid protective vector based on DNA hydrogel according to claim 11, wherein, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

13. A preparation method of the functional nucleic acid protective vector based on DNA hydrogels according to claim 3, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

14. The preparation method of the functional nucleic acid protective vector based on DNA hydrogel according to claim 13, wherein, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

15. A preparation method of the functional nucleic acid protective vector based on DNA hydrogels according to claim 4, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

16. The preparation method of the functional nucleic acid protective vector based on DNA hydrogel according to claim 15, wherein, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

17. A preparation method of the functional nucleic acid protective vector based on DNA hydrogels according to claim 5, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

18. The preparation method of the functional nucleic acid protective vector based on DNA hydrogel according to claim 17, wherein, when the molar ratio of the DNA in the biodegradable high-molecular polymer with DNA-grafted side chain to the cross-linking agent ranges from 8:1 to 2:1, the size of the prepared DNA hydrogel also ranges from 70 nm to 1.3 μm.

19. A preparation method of the functional nucleic acid protective vector based on DNA hydrogels according to claim 6, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.

20. A preparation method of the functional nucleic acid protective vector based on DNA hydrogels according to claim 7, wherein the method comprises the following steps:
(1) the biodegradable polymer with azide groups on its side chain is synthesized by ring opening polymerization, and then the degradable polymer reacts with the diphenylcyclooctyne-modified DNA to obtain a biodegradable high-molecular polymer with DNA-grafted side chain;
(2) the biodegradable high-molecular polymer with DNA-grafted side chain is dissolved into water, and then the functional nucleic acid is added and stirred evenly at room temperature, so that the functional nucleic acid and the biodegradable high-molecular polymer with DNA-grafted side chain are completely paired with each other, after that, the cross-linking agent is added to pair with the biodegradable high-molecular polymer with DNA-grafted side chain completely to obtain DNA hydrogel solution.
